(19) United States
(12) Patent Application Publication
(10) Pub. No.: US 2005/0202079 A1
(43) Pub. Date: Sep. 15, 2005

(54) Novel orally administrable formulation of nitrofurantoin and a method for preparing said formulation

(75) Inventors: Tara Lynn Bielski, Washington, PA (US); Kerry Roy Benson, Morgantown, WV (US)

Correspondence Address: ROTHWELL, FIGG, ERNST & MANBECK, P.C., 1425 K STREET, N.W., SUITE 800, WASHINGTON, DC, 20005, US

(73) Assignee: Mylan Pharmaceuticals Inc., Morgantown, WV (US)

(21) Appl. No.: 10/800,031

(22) Filed: Mar. 15, 2004

(51) Int. Cl. A61K 9/48; A61K 9/20; A61K 31/34

(52) U.S. Cl. 424/451; 424/464; 514/471

(57) ABSTRACT

The present invention relates to a novel orally administrable formulation of nitrofurantoin comprising a first component being a controlled release form, which comprises nitrofurantoin monohydrate, sodium alginate, alginic acid and hypromellose, and a second component being an immediate release form, which comprises macrocrystalline nitrofurantoin. A method for preparing said formulation and a method for treating a bacterial infection using said formulation are also described.

### BACKGROUND OF THE INVENTION

1. Field of the Invention

The present invention relates to a novel orally administrable formulation of nitrofurantoin to treat bacterial infections in a patient, more particularly to a controlled release formulation of nitrofurantoin comprising a controlled release first component and an immediate release second component.

2. Description of Related Art

Nitrofurantoin is a well-known antibacterial compound and has been used extensively as an active ingredient in antibacterial pharmaceutical compositions. Nitrofurantoin has been used successfully for many years for the treatment of urinary tract infections (“UTI”). Its presumed mode of action is based upon its interference with several bacterial enzyme systems. It is rapidly absorbed in the gastrointestinal tract and reaches high concentrations in the urine. A general description of nitrofurantoin is found in D. E. Cadwallader, 15 J. American Pharmaceutical Association 409 (1975); and J. D. Conklin, “The Pharmacokinetics of Nitrofurantoin and Its Related Bioavailability,” 25 Antibiotics and Chemotherapy 233 (1978). Nitrofurantoin is well tolerated in humans, with the most frequent adverse reactions being nausea and emesis.

As with many other pharmaceutical active materials, the pharmacokinetics of nitrofurantoin can be affected by the size and type of the nitrofurantoin crystals used in a dosage form. See, for example, J. K. Haleblian, “Characterization of Habits and Crystalline Modification of Solids and Their Pharmaceutical Applications,” 66 J. Pharmaceutical Sciences 1269 (1975). In particular, the use of macrocrystalline nitrofurantoin has been reported to reduce the level of emetic side effects. However, it has been reported that macrocrystalline nitrofurantoin generally must be administered orally about four times daily to be effective. This is generally inconvenient, especially during the night, which results in difficulties in patient compliance. In addition, such multiple dosing may lead to undesirable fluctuation in the plasma concentration of nitrofurantoin.

Thus, there have been efforts to develop a nitrofurantoin formulation that requires less frequent administration while alleviating such undesirable side effects. For example, U.S. Pat. No. 4,772,473 (“the '473 patent) describes pharmaceutical capsules for oral administration containing a combination of sustained release/rapid release of nitrofurantoin, an embodiment of which has been marketed under the trade name of Macrobid®D. The capsules of the '473 patent comprise separate layers of a first particulate mixture and a second particulate mixture. The first particulate mixture comprises nitrofurantoin, polyvinylpyrrolidone and carboxyvinylpolymer, while the second particulate mixture comprises macrocrystalline nitrofurantoin.

U.S. Pat. No. 4,122,157 (“the '157 patent”) discloses using a hydrophillic gum such as hydroxypropyl methylcellulose, carboxymethylcellulose or polyvinylpyrrolidone to provide slow release of micronized nitrofurantoin by forming a gel barrier. However, the ' 157 patent discourages the use of naturally occurring gums, such as alginic acid, as the hydrophillic gum because of their variable gelation characteristics.

For nitrofurantoin, it is reported that there is a minimum therapeutic concentration of the active drug that must be reached and maintained in certain tissues or the urine of the patient for the desired treatment. Nitrofurantoin is extensively metabolized in the body or is eliminated in the urine over time. In a formulation of a combination of rapid release and sustained release of nitrofurantoin, although a minimum therapeutic concentration of the active drug can be reached by rapid release of nitrofurantoin, a sustained release rate is desired to maintain the minimum therapeutic concentration. Thus, it is desirable to administer a controlled release form of nitrofurantoin in a combined immediate/controlled release formulation to provide a desirable release rate.

### BRIEF SUMMARY OF THE INVENTION

Now it surprisingly has been found that a combination of hypromellose, alginic acid, sodium alginate and nitrofurantoin monohydrate provides a controlled release unit that can maintain a minimum therapeutic concentration of nitrofurantoin over a desired period of time and can be combined in a formulation with an immediate release unit comprising macrocrystalline nitrofurantoin, to provide a combination of an immediate release/controlled release formulation that is effective in treating urinary tract infections with minimal levels of undesired side effects, such as nausea and emesis.

The present invention provides an orally administrable formulation for administering nitrofurantoin to a patient in need thereof which comprises a first component being a controlled release form and a second component being an immediate release form. In accordance with one embodiment of the present invention, the formulation comprises
a) a first component which comprises nitrofurantoin monohydrate, sodium alginate, alginic acid and hypromellose;
b) a second component which comprises macrocrystalline nitrofurantoin; and
c) the formulation provides a therapeutically effective combination of said nitrofurantoin monohydrate and said macrocrystalline nitrofurantoin.

The first and second components can be provided in a single dosage unit or as separate dosage units. In one preferred embodiment, each of said dosage units is provided as at least one tablet. In a further preferred embodiment, said first and second components are encapsulated within a capsule.

Another aspect of the present invention provides an orally administrable formulation for the administration of nitrofurantoin to a patient in need thereof which comprises:
a first component being a controlled release form and a second component being an immediate release form, wherein
a) said first component comprises:
nitrofurantoin monohydrate in an amount of from about 5% to about 50% by weight of said first component;
hypromellose in an amount of from about 5% to about 90% by weight of said first component;
sodium alginate in an amount of from about 2% to about 80% by weight of said first component; and
alginic acid in an amount of from about 2% to about 80% by weight of said first component;
a filler in an amount of from about 2% to about 90% by weight of said first component; and
a lubricant in an amount of from about 0.1% to about 6% by weight of said first component; and
(b) said second component comprises:
macrocrystalline nitrofurantoin in an amount of from about 3% to about 35% by weight of said second component;
a filler in an amount of from about 5% to about 90% by weight of said second component; and
a lubricant in an amount of from about 0.1% to about 6% by weight of said second component; and
and said first and second components are present in a wt:wt ratio of from about 1:1 to about 5:1.

Yet another aspect of the present invention provides an orally administrable formulation for the administration of nitrofurantoin to a patient in need thereof which comprises:
a first component being a controlled release form and a second component being an immediate release form, wherein
(a) said first component comprises:
nitrofurantoin monohydrate in an amount of about 20% by weight of said first component;
hypromellose in an amount of about 20% by weight of said first component;
sodium alginate in an amount of about 20% by weight of said first component; and
alginic acid in an amount of about 20% by weight of said first component;
microcrystalline cellulose in an amount of about 9% by weight of said first component;
dibasic calcium phosphate in an amount of about 10% by weight of said first component; and
magnesium stearate in an amount of about 1% by weight of said first component; and
(b) said second component comprises:
macrocrystalline nitrofurantoin in an amount of about 12.5% by weight of said second component;
lactose in an amount of about 43% by weight of said second component;
microcrystalline cellulose in an amount of about 43% by weight of said second component; and
magnesium stearate with sodium lauryl sulfate in an amount of about 1% by weight of said second component; and
said first and second components are present in a wt:wt ratio of about 2:1.

The present invention further provides a method for treating a bacterial infection in a host which comprises administering to said host a formulation of the present invention. In a preferred embodiment, the formulation is administered twice per day.

These and other advantages and benefits of the present invention will be further appreciated in light of the detailed description of exemplary embodiments below.

### DETAILED DESCRIPTION OF THE INVENTION

As used herein, the term “dosage unit” generally refers to a solid dosage form of a pharmaceutical preparation, which includes, but is not limited to, a tablet and a capsule, preferably a tablet. When there is a single dosage unit in the form of a tablet, the tablet may have a structure comprising an inner, core layer, and an outer layer that encloses or envelopes the inner, core layer. In this embodiment, preferably the first component is in the form of the inner, core layer and the second component is in the form of the outer layer.

The term “tablet” generally is intended to encompass any compressed form of tablet, as, for example, chewable, soluble, effervescent, buccal and subligual tablets.

The formulations of the present invention comprise two separate components, a first component for controlled release and a second component for immediate release of the active pharmaceutical agents contained therein. The two components can be provided in a single dosage unit or as separate dosage units. For example, either or each of the components in the formulation of the present invention can be in the form of granules or powders, which can be compressed into one or more tablets or encapsulated. The components can be layered or mixed or otherwise compounded in a single tablet or a capsule. The order in which the first component and the second component is layered is not critical. The dosage unit can have at least two separate layers, one comprising the first component and the other comprising the second component. Each of the first and second components also can be divided into two or more layers such that multiple layers of either or both of the first and second component could be included in a single dosage unit. In a multilayer embodiment, the first component and the second component are preferably layered in an alternating way. However, it is preferred that each of the first and second components is present in a single layer.

When the two components are provided in a single capsule, they do not have to be provided in the same form. For example, one component can be provided as one or more tablets and the other component as a powder.

Alternatively, the first and second components can be provided as separate dosage units. Each component also can be divided, if desired, into multiple dosage units. Typically, the first component is present in either one or two dosage units, and the second component in a single dosage unit.

Where the first and second components are present as separate dosage units, they are administered to a patient substantially simultaneously, preferably in a capsule that encapsulates the first and second components. When a capsule is used in the present invention, it is preferably soluble in gastrointestinal juice and more preferably in gastric juice. A hard gelatin capsule that is soluble in gastric juice is the most preferred.

The first component of the formulation comprises nitrofurantoin monohydrate, hypromellose, sodium alginate and alginic acid. The second component of the formulation comprises macrocrystalline nitrofurantoin. Each component can further include at least one pharmaceutically acceptable carrier. The carrier can comprise a diluent, lubricant, glidant, surfactant or colorant.

In the present formulation, the first component is preferably present in a weight ratio to the second component of within the range of from about 1:1 to about 5:1, and preferably from about 2:1 to about 3:1, and more preferably about 2:1.

The first component is a controlled release form that generally begins to release the nitrofurantoin monohydrate once the polymers in the component become hydrated to form a matrix. In a preferred embodiment, substantially all of nitrofurantoin monohydrate is released from the first component within about twelve (12) to about fourteen (14) hours after administration of the formulation of the present invention. The second component being an immediate release form generally starts releasing macrocrystalline nitrofurantoin as soon as the formulation reaches the gastrointestinal tract. In a preferred embodiment, substantially all of the macrocrystalline nitrofurantoin is released from the second component within about three (3) hours of administration of the formulation of the present invention.

Nitrofurantoin

As used herein, “nitrofurantoin” is the compound N-(5-nitro-2-furfurylidene)-1-aminohydantoin, or its pharmaceutically acceptable salts, hydrates, or complexes. (See “6445. Nitrofurantoin”, The Merck Index, 10th ed. (1983, pp. 946-947.) Preferred forms of nitrofurantoin for the first and second components are nitrofurantoin monohydrate and macrocrystalline nitrofurantoin, respectively.

A method for preparing nitrofurantoin is disclosed in U.S. Pat. No. 2,610,181, which is incorporated herein by reference. An example of methods for preparing macrocrystalline nitrofurantoin is disclosed in U.S. Pat. No. 3,401,221, which is incorporated herein by reference. “Macrocrystalline nitrofurantoin,” as used herein, generally refers to particulate crystalline nitrofurantoin where at least 90 weight percent of the crystals have a surface area of from about 120 cm²/gm to about 2,000 cm²/gm. Preferred macrocrystalline nitrofurantoin has at least 90 weight percent of the crystals with a surface area of from about 450 cm²/gm to about 2,000 cm²/gm.

For the formulations of the present invention, the first, or controlled release, component comprises from about 25 mg to about 600 mg of nitrofurantoin monohydrate, preferably from about 50 mg to about 300 mg, and more preferably from about 75 mg to about 150 mg. The second, or immediate release, component comprises from about 5 mg to about 400 mg of macrocrystalline nitrofurantoin, preferably from about 12 mg to about 200 mg, and more preferably from about 25 mg to about 100 mg. In a preferred embodiment, nitrofurantoin monohydrate in the first component is present in a weight ratio of about 3:1 to macrocrystalline nitrofurantoin in the second component.

In the formulations of the present invention, nitrofurantoin monohydrate is present from about 5% to about 50% by weight, preferably from about 7% to about 35% by weight, and more preferably from about 10% to about 20% by weight of the first component. Macrocrystalline nitrofurantoin is present from about 3% to about 35% by weight, preferably from about 5% to about 25% by weight, and more preferably from about 10% to about 15% by weight of the second component. The formulation of the present invention provides a therapeutically effective combination of nitrofurantoin monohydrate and macrocrystalline nitrofurantoin.

Hypromellose

Hypromellose is the British Approved Name as well as the recommended International Nonproprietary Name for hydroxypropyl methylcellulose (HPMC). Hypromellose is a cellulose derivative polymer that generally is used to provide a sustained release. When hypromellose is exposed to aqueous fluids, it undergoes rapid hydration and chain relaxation to form a gel of such consistency that drug diffusion through the gel can be controlled.

Hypromellose is commercially available in various grades, under several trade names, including Methocel® E, F, J and K from The Dow Chemical Co., USA, HPM® from British Celanese Ltd. England and Metaluse® SH from Shin Etsu, Ltd, Japan. The various grades available under a given tradename represent differences in methoxyl and hydroxypropoxyl content as well as molecular weight. The methoxyl content ranges from 16.5 to 30 weight % and the hydroxypropoxyl content ranges from 4 to 32 weight %, as determined by the method described in ASTM D-2363-72. All of these various forms of hypromellose are contemplated to be used in the present invention. For example, the present invention contemplates the use of Methocel® K in its various forms having a methoxyl content of 19-24% and a hydroxypropoxyl content of 7-12%, Methocel® E in its various forms, having a methoxyl content of 28-30% to and a hydroxypropoxyl content of 7-12%, and Methocel® F in its various forms having a methoxyl content of 27-30% and a hydroxypropoxyl content of 4-7.5%.

Commercial designations of the various hypromellose are based on the viscosities of 2% aqueous solutions at 20° C. The viscosities range from 3 cps to 100,000 cps and represent number average molecular weights ranging from about 10,000 to over 150,000, as calculated from the data in the “Handbook of Methocel Cellulose Ether Products” (The Dow Chemical Co., 1974). Examples of hypromellose include Metalose® 60 5H50 which is a hypromellose having a hydroxypropoxyl content of 9-12 weight % and a number average molecular weight of less than 50,000; Methocel® E4M, having a 28-30 weight % methoxyl content, a viscosity of 4000 cps, a hydroxy-propoxyl weight % of 7-12 and a number average molecular weight of 93,000; Methocel® E10M, having a viscosity of 10,000 cps, a 28-30 weight % methoxyl content, 7-12 weight % hydroxypropoxyl, Methocel® K4M, having a number average molecular weight of 89,000, viscosity of 4,000,19-24% weight % methoxyl content, and a 7-12 weight % hydroxypropoxyl content; Methocel® KI 5M, having a number average molecular weight of 124,000, a 19-24 weight % methoxyl content and a 7-12 weight % hydroxypropoxyl content; and K100M, having a viscosity of 100,000 cps and a 19-24 weight % methoxyl content and is 7-12 weight % hydroxypropoxyl content, Methocel® J5M, J12M, J20M and J75M, having viscosities of 5,000, 12,000, 20,000, and 75,000, cps, respectively. Various hypromellose materials which can also be used in the first component of the present formulation are described in U.S. Pat. No. 3,870,790, U.S. Pat. No. 4,226,849, U.S. Pat. No. 4,357,469, U.S. Pat. No. 4,369,172, U.S. Pat. No. 4,389,393, U.S. Pat. No. 4,259,314, U.S. Pat. No. 4,540,566, U.S. Pat. No. 4,556,678, the contents of all of which are incorporated herein by reference.

Hypromellose is present in the first component of the present formulation in an amount of from about 5% to about 90% by weight, preferably from about 5% to about 60% by weight, more preferably from about 10% to about 30% by weight, and most preferably about 20% by weight of the first component.

Alginic Acid and Sodium Alginate

Alginic acid is a natural acidic polysaccharide extracted from so-called brown algae (Phaecophyceae) with a high molecular weight varying between about 20,000 and 240,000, and containing chains formed by D-mannuronic acid and L-guluronic acid. The degree of polymerization varies according to the type of algae used for extraction, the season in which the algae were gathered and the place of origin of the algae, as well as the age of the plant itself. The main species of brown algae used to obtain alginic acid are, for example, Macrocystis pyrifera, Laminaria cloustoni, Laminaria hyperborea, Laninaria flexicaulis, Laninaria digitata, Ascophyllum nodosum, and Fucus serratus.

Alginic acid is found in these algae as an extensive constituent of the cell walls in the form of a mixture of some of its alkaline salts, especially sodium salt. This mixture is also known as “algin.” These salts are normally extracted in aqueous conditions with a sodium carbonate solution and it is possible to obtain alginic acid directly from this extract by precipitation with an acid, for example a mineral acid such as hydrochloric acid. An indirect preparation procedure involves first making an insoluble calcium salt by adding a soluble calcium salt, such as chloride, and after washing this salt, alginic acid is obtained again by treatment with an acid. Alginic acid or sodium alginates however, also can be obtained microbiologically, for instance by fermentation with Pseudomonas aeruoinosa or mutants of Pseudomonas putida, Pseudomonas fluorescens or Pseudomonas miendocina.

Alginic acid or sodium alginate can have a viscosity of from about 4 to about 8,000, preferably from about 35 to about 1300, and more preferably from about 65 to about 400. Examples of commercially available alginic acid and sodium alginate, which are contemplated to be used in the present invention, include Kelacid® (alginic acid), Manucol® LKX (sodium alginate), Keltone® LVCR (sodium alginate) and Keltone® HVCR (sodium alginate), all of which are from International Specialty Products, USA.

Each of alginic acid and sodium alginate is present in the first component of the present formulation in an amount of from about 2% to about 80% by weight, perferably from about 5% to about 60% by weight, more preferably from about 10% to about 30% by weight, and most preferably about 20% of the first component.

Pharmaceutically Acceptable Carriers

The formulations of the present invention typically can comprise at least one pharmaceutically acceptable carrier in each of the first and second components. The term “pharmaceutically acceptable carriers,” as used herein generally refers to one or more inert materials that are added either to impart satisfactory processing characteristics to the formulation or to give additional desirable physical characteristics to the formulation. These materials include, without limitation, a diluent or a filler, a binder, a glidant, a lubricant, a colorant, an antioxidant, a flavoring or sweetening agent and other materials known in the art. By “inert,” as used herein, is meant a material that is capable of being comingled without interacting in a manner which would substantially affect the pharmaceutical efficacy of the formulation under ordinary use situations.

In one embodiment, either or both of the first and second components of the present formulation can contain a lubricant. Examples of a lubricant include stearate salts, such as alkaline earth and transition metal salts thereof, such as calcium, magnesium, or zinc; sodium stearyl fumarate; stearic acid; polyethylene oxide; talc; hydrogenated vegetable oil and vegetable oil derivatives; silica; silicones; high molecular weight polyalkylene glycol, such as high molecular weight polyethylene glycol; monoesters of propylene glycol; and saturated fatty acid containing about 8-22 carbon atoms and preferably 16-20 carbon atoms. Stearate salts, stearic acid and talc are preferred, and magnesium stearate with or without a surfactant such as sodium lauryl sulfate is more preferred. In a dosage unit of the first or second component, the lubricant is used in a lubricating effective amount ranging from about 0.1% to about 6% by weight, preferably from about 0.3% to about 3% by weight, more preferably about 0.5% to about 1.5% by weight, and the most preferably about 1% by weight of the first or second component.

Another pharmaceutically acceptable carrier that can be added to the present formulation is a diluent or a filler (“diluent”). A diluent can be water soluble or water insoluble. Examples of a diluent include, without limitation, calcium salts, such as calcium sulfate, dicalcium phosphate, tricalcium phosphate, calcium lactate, or calcium gluconate; glycerol phosphate; citrates; sucrose; starch; mannitol, dextrose; lactose; microcrystalline cellulose; fructose; xylitol; sorbitol; and mixtures thereof. The diluent, if present in the first component, is present in an amount ranging from about 8% to about 38% by weight, preferably from about 15% to about 25%, and more preferably from about 18% to about 20%. The diluent can be present in the second component in an amount ranging from 75% to about 95%, preferably from about 80% to about 90%, and more preferably from about 85% to about 87%. In one embodiment, a combination of microcrystalline cellulose and dibasic calcium phosphate are used in the first component and microcrystalline cellulose and lactose in the second component. For the first component, microcrystalline cellulose can be added in an amount ranging from about 2% to about 90% by weight, preferably from about 3% to about 60% by weight, more preferably from about 5% to about 20% by weight, and most preferably about 9% by weight of the first component. Dibasic calcium phosphate can be used in an amount ranging from about 2% to about 90% by weight, preferably from about 5% to about 60% by weight, more preferably from about 10% to about 30% by weight, and most preferably about 10% by weight of the first component. For the second component, each of microcrystalline cellulose and lactose can be added in an amount ranging from about 5% to about 90% by weight, preferably from about 10% to about 70% by weight, more preferably from about 20% to about 50% by weight, and most preferably about 43% by weight of the second component.

As other optional carriers, the present formulation can contain a colorant in either the first or second component. The colorants include, but are not limited to, various food colors, e.g., iron oxide, FD & C colors, such as FD & C Yellow No. 6, food lakes and the like. Other optional ingredients that can be used in the present formulation include, without limitation, preservatives such as methyl parabeans; artificial sweeteners such as saccharin sodium, aspartame, dipotassium glycyrrhizinate, stevia, thaumatin and the like; flavorants such as lemon, lime, orange and menthol; and antioxidizing agents such as ascorbic acid, sodium metabisulphite. These optional ingredients, if present, are preferably present in amounts ranging from about 0.05% to about 1.5% by weight, and more preferably about 0.5% by weight of the first or second component.

A preferred formulation of the present invention includes the following:

The first component:

| Ingredient | % by weight of the first component |
|---|---|
| Nitrofurantoin monohydrate | 20.0 |
| Hypromellose | 20.0 |
| Sodium alginate | 20.0 |
| Alginic acid | 20.0 |
| Microcrystalline cellulose | 9.0 |
| Diabasic calcium phosphate | 10.0 |
| Magnesium stearate | 1.0 |

The second component:

| Ingredient | % by weight of the second component |
|---|---|
| Macrocrystalline nitrofurantoin | 12.5 |
| Lactose | 43.0 |
| Microcrystalline cellulose | 43.1 |
| FD&C Yellow No. 6 Lake HT | 0.4 |
| Magnesium stearate/SLS | 1.0 |

The first and second components of the formulation of the present invention are prepared in accordance with procedures well-known in the art. Where the first and second components are present in the form of separate tablets, in one embodiment all of the ingredients, except for the lubricant and diluent, for the first component, which comprise nitrofurantoin monohydrate, sodium alginate, alginic acid, hypromellose, and any desired pharmaceutically acceptable carriers, are mixed with a sufficient amount of granulating solvent to form a substantially uniform granulation. The granulating vehicle is one that is inert with the components and has a low boiling point, e.g., preferably less than about 120° C. It is preferably a solvent that contains hydroxyl groups, such as water or an alcohol containing 1-4 carbon atoms, e.g., isopropyl alcohol or ethanol. An aqueous dispersion also can be utilized. In a preferred embodiment, the granulating vehicle is water or alcohol.

The substantially uniform granulation is dried. In this step, the solvent is removed from the granulation by physical means known to the skilled artisan, e.g., by evaporation. The resulting granules are milled, e.g., passed through a screen or sieve to further reduce the size of the particles to the desired size. The milled granules are then blended with a pharmaceutically acceptable carrier including a lubricant and diluent to produce a uniform blend, i.e., homogenous with respect to the drug. The resulting blend then can be compressed to form a tablet. In a preferred embodiment, the ingredients are granulated and immediately dried such as by using a fluid bed granulation process.

The formation of the second component is well within the purview of a person skilled in the art of formulation of pharmaceutical composition. In a preferred embodiment, the second component is prepared by blending macrocrystalline nitrofurantoin, lactose and microcrystalline cellulose. The material can be milled to ensure homogeneity. The resulting material then is blended with magnesium stearate/sodium lauryl sulfate in a blender such as a V-blender. The final blended materials then can be compressed into a tablet.

The first and second components prepared as separate dosage units can be administered in a substantially simultaneous way, with or without further formulating into a single formulation. In a preferred embodiment, the first and second components are encapsulated into a capsule, preferably using a hand-filling or standard pharmaceutical encapsulation machines.

Where the first and second components are present in a single dosage unit, in one embodiment each of the final blended materials for the first and second components is prepared in accordance with the wet granulation and direct compression steps described above, and then is compressed into a multilayer tablet or compressed on a tablet machine to produce a tablet-in-tablet (or covered tablet or compression coated tablet). This tablet may be filled into a capsule, if desired. In another embodiment, each of the final blended materials for the first and second components is prepared in accordance with the wet granulation and direct compression steps described above, and then is encapsulated into a capsule in separate layers, preferably using a hand-filling or standard pharmaceutical encapsulation machine.

Each of the first and second components can be coated with a pharmaceutically acceptable coating material normally used in the pharmaceutical industry. The final formulation such as a tablet or capsule can be coated with a pharmaceutically acceptable coating material normally used in the pharmaceutical industry, if desired.

The formulation of the present invention, thus formed using the selected polymers, provides a desired drug release profile for nitrofurantoin. More specifically, it has a predetermined, controlled and sustained action and a desired controlled pattern so that a minimum concentration of nitrofurantoin is maintained for about twelve (12) hours, which permits administration of the formulation twice per day.

The formulation of the present invention comprising nitrofurantoin can be used to treat a patient suffering from a bacterial infection, more particularly urinary tract infections (“UTI”). The present formulation is particularly effective in treating UTI caused by certain strains of Escherichia coli or Staphylococcus saprophyticus.

The present formulation is administered in a therapeutically effective amount. Suitable dosage ranges are well recognized in the art, or can readily be determined by routine adjustment. Preferably the formulation is administered in an amount of 100 mg of nitrofurantoin two times per day for a period of about 7 days. The precise amount of drug administered to a patient will depend on a number of factors, including the age of the patient, the severity of the condition and the past medical history, among other factors, and will always lie within the sound discretion of the administering physician.

The invention will be further described with respect to the following examples; however, the scope of the invention is not limited thereby. All percentages in this specification, unless otherwise specified, are by weight.

### EXAMPLE

### Example 1

The first component with the following composition was prepared:

| Ingredient | mg per tablet | % |
|---|---|---|
| Purified Water, USP | 150.0 | — |
| Nitrofurantoin Monohydrate | 40.125 | 20.0 |
| (Water Factor 1.07 | | |
| Hypromellose | 40.125 | 20.0 |
| (Methocel K100MP) | | |
| Microcrystalline Cellulose | 17.75 | 9.0 |
| (Avicel PH 102) | | |
| Alginic Acid | 40.0 | 20.0 |
| (Kelacid) | | |
| Sodium Alginate | 40.0 | 20.0 |
| (Manucol LKX) | | |
| Dibasic Calcium Phosphate, | 20.0 | 10.0 |
| Anhydrous | | |
| (A-Tab) | | |
| Magnesium Stearate | 2.0 | 1.0 |
| Total | 200 | 100 |

Pre-determined amounts of Avicel PH 102, Nitrofurantoin Monohydrate, Manucol LKX, Methocel K100MP, and Kelacid were fluidized in a fluid bed for three (3) minutes to mix the materials. The blended materials then were top spray granulated with a pre-determined portion of purified water at a spray rate of 1000 g/min (approx. 15 minutes) with an inlet temperature of 45° C. and an atomizing air pressure of 50 PSI. The granulated material then was dried in the fluid bed at an inlet temperature of 70° C. for approximately 60 minutes. The dried granulation was milled through a Fitzmill using knives forward, medium speed and a #1B screen (0.050”). The magnesium stearate and A-Tab were individually screened through a #18 mesh screen. The milled granulation, magnesium stearate, and A-tab then were blended in a V-blender for 15 minutes. The final blended material was compressed on a tablet press at a target weight of 200 mg.

### Example 2

The second component with the following composition was prepared:

| | mg per | |
|---|---|---|
| Ingredient | tablet | % |
| Nitrofurantoin Macrocrystals | 25.0 | 12.5 |
| Microcrystalline Cellulose | 86.2 | 43.1 |
| (Avicel PH 200) | | |
| Lactose, Monohydrate | 86.0 | 43.0 |
| (Fast Flo) | | |
| FD&C Yellow #6 Lake HT (15-18%) | 0.8 | 0.4 |
| Magnesium Stearate/Sodium Lauryl | 2.0 | 1.0 |
| Sulfate (94/6) | | |
| (Stear-O-Wet M) | | |
| Total | 200 | 100 |

Pre-determined amounts of lactose, nitrofurantoin macrocrystals, FD&C yellow #6, and Avicel PH 200 were blended in a V-blender for 15 minutes. The blended material was milled using a Comil equipped with a 032R screen at 700 RPM. The magnesium stearate/SLS was screened through a #30 mesh screen. The milled material and the magnesium stearate/SLS were blended in a V-blender for 15 minutes. The final blended material was compressed on a tablet press at a target weight of 200 mg. One tablet thus prepared and two tablets of the first component as prepared above in Example 1 were filled into a hard gelatin capsule using an encapsulation machine.

1. An orally administrable formulation for administering nitrofurantoin to a patient in need thereof which comprises:
a first component being a controlled release form and a second component being an immediate release form,
wherein
(a) said first component comprises nitrofurantoin monohydrate, sodium alginate, alginic acid and hypromellose;
(b) said second component comprises macrocrystalline nitrofurantoin; and
(c) said formulation provides a therapeutically effective combination of said nitrofurantoin monohydrate and said macrocrystalline nitrofurantoin.

2. The formulation of claim 1, wherein each of said first and second components independently is in the form selected from the group consisting of granules and powders and wherein said components are provided in a single dosage unit.

3. The formulation of claim 2, wherein said dosage unit is a tablet.

4. The formulation of claim 3, wherein said first and second components are present as separate layers.

5. The formulation of claim 2, wherein said dosage unit is a capsule.

6. The formulation of claim 1, wherein each of said components is in the form of one or more tablets and said tablets are encapsulated within a single capsule.

7. The formulation of claim 5, wherein each of said components in said capsule is in the form of granules or powders and said components are present as separate layers.

8. The formulation of claim 5, wherein one of said components in said capsule is in the form of at least one tablet and the other of said components in said capsule is in the form of granules or powders.

9. The formulation of claim 1, wherein each of said first and second components is provided as a separate dosage unit.

10. The formulation of claim 9, wherein each component is provided as at least one tablet.

11. The formulation of claim 1, wherein said first component comprises from about 25 mg to about 600 mg nitrofurantoin monohydrate.

12. The formulation of claim 11, wherein said first component comprises from about 50 mg to about 300 mg nitrofurantoin monohydrate.

13. The formulation of claim 12, wherein said first component comprises from about 75 mg to about 150 mg nitrofurantoin monohydrate.

14. The formulation of claim 11, wherein said second component comprises from about 5 mg to about 400 mg macrocrystalline nitrofurantoin.

15. The formulation of claim 14, wherein said second component comprises from about 12 mg to about 200 mg macrocrystalline nitrofurantoin.

16. The formulation of claim 15, wherein said second component comprises from about 25 mg to about 100 mg macrocrystalline nitrofurantoin.

17. The formulation of claim 1, wherein from about 5% to about 90% by weight of said first component is hypromellose.

18. The formulation of claim 17, wherein from about 5% to about 60% by weight of said first component is hypromellose.

19. The formulation of claim 18, wherein from about 10% to about 30% by weight of said first component is hypromellose.

20. The formulation of claim 1, wherein from about 2% to about 80% by weight of said first component is sodium alginate.

21. The formulation of claim 20, wherein from about 5% to about 60% by weight of said first component is sodium alginate.

22. The formulation of claim 21, wherein from about 10% to about 30% by weight of said first component is sodium alginate.

23. The formulation of claim 1, wherein from about 2% to about 80% by weight of said first component is alginic acid.

24. The formulation of claim 23, wherein from about 5% to about 60% by weight of said first component is alginic acid.

25. The formulation of claim 24, wherein from about 10% to about 30% by weight of said first component is alginic acid.

26. The formulation of claim 1, wherein said first component comprises
nitrofurantoin monohydrate in an amount of about 20% by weight of said first component;
hypromellose in an amount of about 20% by weight of said first component;
sodium alginate in an amount of about 20% by weight of said first component; and
alginic acid in an amount of about 20% by weight of said first component; and
said second component comprises macrocrystalline nitrofurantoin in an amount of about 12.5% by weight of said second component; and
said first component and said second component are present in a wt:wt ratio of about 1:1 to about 5:1.

27. The formulation of claim 1, wherein each of said first and second components further comprises at least one pharmaceutically acceptable carrier, provided that said carrier in said first component does not comprise polyvinylpyrrolidone or carboxyvinylpolymer.

28. The formulation of claim 27, wherein said carrier comprises a diluent, lubricant, surfactant, glidant or colorant.

29. The formulation of claim 1 or 26, wherein at least one of said first and second components is coated.

30. An orally administrable formulation for the administration of nitrofurantoin to a patient in need thereof which comprises:
a first component being a controlled release form and a second component being an immediate release form, wherein
(a) said first component comprises:
nitrofurantoin monohydrate in an amount of from about 5% to about 50% by weight of said first component;
hypromellose in an amount of from about 5% to about 90% by weight of said first component;
sodium alginate in an amount of from about 2% to about 80% by weight of said first component;
alginic acid in an amount of from about 2% to about 80% by weight of said first component;
a diluent in an amount of from about 2% to about 90% by weight of said first component; and
a lubricant in an amount of from about 0.1% to about 6% by weight of said first component; and
(b) said second component comprises:
macrocrystalline nitrofurantoin in an amount of from about 3% to about 35% by weight of said second component;
a diluent in an amount of from about 5% to about 90% by weight of said second component; and
a lubricant in an amount of from about 0.1% to about 6% by weight of said second component; and
said first and second components are present in a wt:wt ratio of from about 1:1 to about 5:1.

31. The formulation of claim 30, wherein said first or second component further comprises a colorant.

32. The formulation of claim 31, wherein said colorant is present in an amount of about 0.5% by weight of said second component.

33. The formulation of claim 30, wherein said first and second components are present in a wt:wt ratio of about 2:1.

34. The formulation of claim 30, wherein said diluent comprises microcrystalline cellulose, dibasic calcium phosphate, lactose, starch, sucrose or mannitol.

35. The formulation of claim 30, wherein said lubricant comprises magnesium stearate, talc, calcium oxide, zinc oxide, stearic acid, sodium stearyl fumarate or vegetable oil.

36. The formulation of claim 30, wherein at least one of said first and second components is coated.

37. An orally administrable formulation for the administration of nitrofurantoin to a patient in need thereof which comprises:
a first component being a controlled release form and a second component being an immediate release form, wherein
(a) said first component comprises:
nitrofurantoin monohydrate in an amount of about 20% by weight of said first component;
hypromellose in an amount of about 20% by weight of said first component;
sodium alginate in an amount of about 20% by weight of said first component; and
alginic acid in an amount of about 20% by weight of said first component;
microcrystalline cellulose in an amount of about 9% by weight of said first component;
dibasic calcium phosphate in an amount of about 10% by weight of said first component; and
magnesium stearate in an amount of about 1% by weight of said first component; and
(b) said second component comprises:
macrocrystalline nitrofurantoin in an amount of about 12.5% by weight of said second component;
lactose in an amount of about 43% by weight of said second component;
microcrystalline cellulose in an amount of about 43% by weight of said second component; and
magnesium stearate with sodium lauryl sulfate in an amount of about 1% by weight of said second component; and
said first and second components are present in a wt:wt ratio of from about 1:1 to about 5:1.

38. The formulation of claim 37, wherein said first or second component further comprises a colorant.

39. The formulation of claim 38, wherein said colorant is present in an amount of about 0.5% by weight of said second component.

40. The formulation of claim 37, wherein said first and second components are present in a wt:wt ratio of about 2:1.

41. The formulation of claim 30 or 37, wherein each of said first and second components independently is in the form selected from the group consisting of granules and powders and wherein said components are provided in a single dosage unit.

42. The formulation of claim 41, wherein said dosage unit is a tablet.

43. The formulation of claim 42, wherein said first and second components are present as separate layers.

44. The formulation of claim 37, wherein said dosage unit is a capsule.

45. The formulation of claim 37, wherein each of said components is in the form of one or more tablets and said tablets are encapsulated within a single capsule.

46. The formulation of claim 44, wherein each of said components in said capsule is in the form of granules or powders and said components are present as separate layers.

47. The formulation of claim 44, wherein one of said components in said capsule is in the form of at least one tablet and the other of said components in said capsule is in the form of granules or powders.

48. The formulation of claim 30 or 37, wherein each of said first and second components is provided as a separate dosage unit.

49. The formulation of claim 48, wherein each component is provided as at least one tablet.

50. A method for preparing a formulation which comprises
(a) admixing nitrofurantoin monohydrate, hypromellose, alginic acid sodium alginate and at least one pharmaceutically acceptable carrier and forming said admixture into a controlled release first component; and
(b) admixing macrocrystalline nitrofurantoin and at least one pharmaceutically acceptable carrier and forming said admixture to form an immediate release second component.

51. The method of claim 50, further comprising encapsulating said first and second components.

52. The method of claim 50, wherein each of said first and second components independently is in the form selected from the group consisting of granules and powders and wherein said components are formed into a single dosage unit.

53. The method of claim 50, wherein said dosage unit is a tablet.

54. The method of claim 53, wherein said first and second components are present as separate layers.

55. The method of claim 52, wherein said dosage unit is a capsule.

56. The method of claim 50, wherein each of said components is in the form of one or more tablets and said tablets are encapsulated within a single capsule.

57. The method of claim 55, wherein each of said components in said capsule is in the form of granules or powders and said components are present as separate layers.

58. The method of claim 55, wherein one of said components in said capsule is in the form of at least one tablet and the other of said components in said capsule is in the form of granules or powders.

59. The method of claim 50, wherein each of said first and second components is formed into a separate dosage unit.

60. The method of claim 59, wherein each component is provided as at least one tablet.

61. A method for treating a bacterial infection in a host which comprises administering to said host said formulation of claim 1, 30 or 37.

62. The method of claim 61, wherein said first component and said second component of said formulation are present in a wt:wt ratio of about 2:1 to about 3:1.

63. The method of claim 62, wherein said first component and said second component are present in a wt:wt ratio of about 2:1.

64. The method of claim 61, wherein nitrofurantoin monohydrate and macrocryststalline nitrofurantoin are present in a combined weight of about 100 mg.

65. The method of claim 64, wherein said formulation is administered two times per day for a period of about 7 days.

66. The formulation of claim 37, wherein at least one of said first and second components is coated.
